(19) United States
(12) Patent Application Publication
(10) Pub. No.: US 2016/0000715 A1
(43) Pub. Date: Jan. 7, 2016

(54) Combinations of Oral Medicaments Bonded by a Wrapping

(71) Applicant: DUO-GE, PARIS (FR)

(72) Inventors: Anne-Emmanuelle LUGRIN, ANTIBES (FR); Claude GRISCELLI, PARIS (FR); Mehdi EL GLAOUI, GENEVE (CH); Guillaume EL GLAOUI, PARIS (FR); Jean HOFFELT, LA GARENNE COLOMBES (FR)

(21) Appl. No.: 14/678,464

(22) Filed: Apr. 3, 2015

Related U.S. Application Data
(62) Division of application No. 12/864,397, filed on Oct. 12, 2010, now abandoned.
(60) Provisional application No. 61/006,651, filed on Jan. 25, 2008.
(60) Provisional application No. 61/006,652, filed on Jan. 25, 2008.

(30) Foreign Application Priority Data
Jan. 25, 2008 (FR) 08 50460
Jan. 25, 2008 (FR) 08 50461

(51) Int. Cl. A61K 9/24; A61J 3/06; A61K 31/196; A61K 31/366; A61K 31/235; A61K 31/401; A61K 31/585; A61K 31/138; A61K 31/4439; A61K 31/7048; A61K 31/43; A61K 31/192; A61K 31/675; A61K 31/155; A61K 31/704; A61K 31/455; A61K 31/167; A61K 31/616; A61K 31/4365; A61K 33/14; A61K 31/573; A61K 31/63; A61K 31/451; A61K 31/345; A61K 31/55; A61K 31/5517; A61K 9/28

(52) U.S. Cl. CPC A61K 9/209; A61K 9/284; A61J 3/06; A61K 9/2893; A61K 31/196; A61K 31/366; A61K 31/235; A61K 31/401; A61K 31/585; A61K 31/138; A61K 31/4439; A61K 31/7048; A61K 31/43; A61K 31/192; A61K 31/675; A61K 31/155; A61K 31/704; A61K 31/455; A61K 31/167; A61K 31/616; A61K 31/4365; A61K 33/14; A61K 31/573; A61K 31/63; A61K 31/451; A61K 31/345; A61K 31/55; A61K 31/5517

(57) ABSTRACT

An oral pharmaceutical dosage form is provided which contains at least two medicaments, in which form the medicaments on the one hand are brought together in a leakproof and in-vivo water soluble wrapping and on the other hand are separated so that the active principle of the combined medicaments cannot come into contact with one another. At least one of the medicaments can be chosen from the following therapeutic classes: non-steroidal anti-inflammatory drug (NSAID), proton pump inhibitor (PPI), beta-blocker, statin, conversion enzyme inhibitor (CEI), biguanide, myorelaxant, calcium inhibitor, corticoid, antidepressant, benzodiazepine, non-atropine-like intestinal transit retarder, intestinal antibacterial, and the following therapeutic molecules: spironolactone, propranolol, clarithromycin, amoxycillin, low-dose acetylsalicylic acid, potassium, and clopidogrel.

The present invention relates to a novel oral pharmaceutical dosage form and to oral pharmaceutical combinations which are presented as said novel pharmaceutical dosage form and the purpose of which is to propose a novel therapeutic treatment method.

The large majority of proprietary medicines comprise a single active principle. The administration of several active principles to a patient therefore involves administering as many proprietary medicines.

From the patient's point of view, this gives rise to the problem of so-called compliance, and this question is becoming ever more problematic owing to the multiplication of coprescriptions, especially in the elderly.

The doctor himself is faced with the difficulty of coprescribing under the best conditions of effectiveness and safety, in so far as coprescriptions by the doctor are carried out somewhat empirically, especially in terms of population, posology and dosage.

The combination of a plurality of active principles in the same galenical form has been considered. Gelatin capsules having two separate compartments or gelatin capsules nested one inside the others (FR 2524311, US 2005/053648) have been proposed, but these solutions have not been put into practice, doubtless because of expected difficulties in terms of handling, especially filling, handling of the separating caps, returns for filling and closure of the compartments.

Even more complex and specific forms have been conceived, as in WO01/08666, which describes assemblies provided with interconnection means which are to be fastened by welding.

Other techniques have been described, especially in FR2335206, which describes a pharmaceutical dosage form in which the medicaments are deposited (by spraying or electrostatically) on continuous edible strips of paper and/or of polymer material. The galenical form comprises a plurality of layers of edible sheets, which can carry one or more active principles, the layers or sheets are so arranged that they do not exhibit active principle on the surface that is to constitute the outer surface of the galenical form, and they are sealed so as to trap the active principle(s) inside.

U.S. Pat. No. 5,897,910 aims to make the production of film-coated tablets simpler and more economical. It describes a method for the production of film-coated tablets in which the formation of the tablet and the film coating thereof with the aid of a sheet of material are carried out simultaneously.

In respect of tablets, multilayers, for example in WO97/25064, especially bilayers, have also been proposed. However, multilayer technology is also difficult to implement if the dosage of each medicament is to be respected very precisely, given the problems of stability and expiry of medicaments.

It is an object of the invention, therefore, to remedy those disadvantages by proposing a novel oral pharmaceutical dosage form which brings together two or more medicaments and which is simple to manufacture.

Another object of the invention is to remedy the above-described disadvantages by proposing a combination of medicaments in a novel pharmaceutical dosage form which is simple to manufacture.

Another object of the invention is to provide such a pharmaceutical dosage form which is likely to simplify the procedures of registration as a medicament.

Another object of the invention is to provide such a form which is especially designed to correspond to very precise dosages, posologies and patients and simplify the work of prescribing for the doctor.

Yet another object of the invention is to propose a solution which allows the dosage of each of the combined medicaments to be made certain.

Yet another object of the invention is to propose a solution which makes it possible not to modify the bioavailability of the combined medicaments.

Yet another object of the invention is to propose a versatile solution which allows different galenical forms or forms having different weights or volumes to be combined.

Those objects are achieved by the invention, which relates to an oral pharmaceutical dosage form containing at least two medicaments, in which form the medicaments on the one hand are brought together in a leakproof water-soluble wrapping or envelop and on the other hand are separated so that the active principles of the combined medicaments cannot come into contact with one another.

Within the scope of the invention, water-soluble means that the wrapping is dissolvable and dissolved within a cavity of the digestive system, e.g. where absorption of the combined medicaments is desired, it being possible for the cavity to be the mouth, the stomach or the intestine. The cavity may be the stomach, it being possible for the medicaments to be absorbed in the stomach or in the intestine, including at least one in the stomach and at least one in the intestine. In a particular embodiment, the wrapping dissolves rapidly and particularly instantaneously in water or in saliva. The wrapping is said to be water-soluble in vivo.

The wrapping is called leakproof in order to isolate the medicaments from the ambient medium. Especially, the wrapping isolates the medicaments from ambient moisture. Isolation can be improved in the manner known per se by the packaging of the pharmaceutical dosage form, for example in a blister or any other known means. The wrapping protects the pharmaceutical dosage form until it is ingested by the patient.

The medicaments are preferably separated by a sheet or wall which extends inside the wrapping. The wrapping and the sheet or wall, where the last is present, are, of course, made of pharmaceutical (pharmaceutically acceptable) material. The wrapping and the sheet or wall are in contact in order to provide continuity of separation and protection.

Within the scope of the invention, a medicament is understood as being a galenical or galenic formulation (or dosage form) of an active principle, that is so say the mixture of the active principle and one or more excipients, adjuvants, carriers, substrates, in a solid oral galenical form. The medicaments or galenical forms are preconstituted. A solid oral galenical form is understood as being a solid oral form of administration, that is to say a form which is conventionally suitable for administration such as by the oral route. This includes especially the forms of tablet, coated tablet, gelatin capsule or soft capsule. This excludes a powdered or liquid composition which would not be included in a solid oral galenical form. After dissolution of the wrapping, the various galenical formulations or medicaments are released into a body cavity in their original form, as if each of the medicaments had been administered separately from the other(s).

According to a feature of the invention, the dosages of the medicaments so combined are coordinated. This means that the dosages of the associated medicaments are optimised according to an optimum therapeutic effectiveness in a single administration.

The oral pharmaceutical dosage form of the invention can comprise especially, without being limited, two, three or four medicaments.

The pharmaceutical dosage forms according to the invention can have one, a plurality or all of the following features:
the medicaments are commercial galenical forms;
the medicaments are galenical forms similar to commercial galenical forms in the sense that they have the same qualitative and quantitative composition but a different geometric form;
combination of at least one commercial galenical form and at least one similar galenical form;
the wrapping and optionally the separating sheet or wall is made of material that is water-soluble under the pH conditions of the predetermined absorption site; this can be the mouth, the stomach or the intestine; the separating sheet or wall, unlike the wrapping, need not necessarily be made of water-soluble material; it must, however, be made of pharmaceutical material that is compatible with the material of the wrapping; it is therefore more convenient to use the same material;
the water-soluble material is water-soluble at the pH of the mouth (of saliva), that is to say a pH of the order of from 7 to 8;
the water-soluble material is water-soluble at the pH of the stomach (very acidic pH, generally about 1);
the water-soluble material is water-soluble at the pH of the intestine, that is to say an alkaline pH (about 8 to 9);
the combined medicaments are to be absorbed in the same cavity of the digestive system, or in different cavities;
the pharmaceutical dosage form is limited to a total weight of medicament of less than or equal to 1500 mg, preferably less than or equal to 1000 mg, especially less than or equal to 800, 700 or 600 mg, in particular less than or equal to 500 or 400 mg;
the pharmaceutical dosage form has a total volume which is compatible with easy swallowing for forms other than those which disintegrate in the mouth;
the galenical forms of the combined medicaments are of the same nature; for example selected from tablets, coated tablets, gelatin capsules or soft capsules;
the galenical forms of the combined medicaments are of different natures, for example for the combination of two medicaments: tablet and coated tablet, tablet and gelatin capsule, tablet and soft capsule, and gelatin capsule, coated tablet and soft capsule, soft capsule and gelatin capsule;
in the case especially where at least one of the medicaments is a capsule or a gelatin capsule, the separating sheet or wall can be omitted, physical separation in this case being ensured by the wall of the gelatin capsule or capsule;
the weight of the combined medicaments is substantially identical or is different;
the volume of the combined medicaments is substantially identical or is different.

According to a first embodiment, the wrapping is of the soft capsule type and is obtained from two half-capsules of gelatin or an analogous material, which are sealed together around the combined medicaments, preferably with separation by a separating sheet or wall (or more depending on the number of medicaments), the latter preferably being made from the same material as the capsule, for example of gelatin or the like. The production process for a two-in-one form can comprise the use of two conventional honeycomb rollers. The counter-rotating rollers are juxtaposed in a manner known per se. The rollers are supplied with three strips of gelatin or the like (two for the wrapping and a central one for the separation) and with the two medicaments. Sealing of the strips of gelatin or the like is carried out by the pressure exerted between the two rollers.

According to a second embodiment, the wrapping is a matrix of rice paper or analogous material, especially a matrix formed by one part, for example an oblong part, having at least two compartments, each of which receives a medicament, and one part forming a cover. Closing can be effected by simple nesting or by bonding, for example wet bonding according to the method conventional for bonding that material.

According to a third embodiment, the wrapping is an inclusion matrix, the medicaments being included in that matrix. The method can consist in bringing the medicaments together in a mould into which a liquid binder is poured in an appropriate manner, optionally in two phases, which binder, after curing, will form a matrix that is water-soluble under the appropriate pH conditions. The matrix can especially be made of gelatin or the like or alternatively of a polymeric material such as a polyethylene glycol, for example PEG 6000.

According to a fourth embodiment, which is preferred, the wrapping is formed by two sheets of water-soluble material which are applied so as to surround the combined medicaments. They are then sealed to one another.

In this embodiment, it is preferred to use one or more sheet(s), preferably of the same material, to separate the medicaments from one another inside the wrapping. The sheet or sheets can advantageously be sealed to the edges of the sheets of the wrapping at the same time as the edges are sealed to one another.

Accordingly, in a preferred form of this embodiment, the pharmaceutical dosage form comprises at least two medicaments, for example two, a wrapping produced from two sheets of pharmaceutical material and at least one (if there are two medicaments, or one or two if there are three medicaments, etc.) separating sheet made of pharmaceutical material, the three sheets being sealed together over the entire periphery of the pharmaceutical dosage form.

The sheets of pharmaceutical material for forming the wrapping and optionally the separating sheets or walls can have one, a plurality or all of the following features:
they are of the same nature for the separating sheet and the sheets of the wrapping;
they are sealed at their edges;
they are water-soluble at the pH of the predetermined absorption site;
they are water-soluble at the pH of the mouth (saliva), that is to say a pH of the order of from 7 to 8;
they are water-soluble at the pH of the stomach (very acidic pH, generally about 1);
they are water-soluble at the pH of the intestine, that is to say an alkaline pH (pH approximately 8 to 9);
they are made of pharmaceutical material for oral ingestion;
they are formed of or comprise one of the following materials: biomaterials, especially biomaterials obtained from or based on algae, cellulose polymers or copolymers, polyvinyl alcohol, lactic and/or glycolic acid derivatives, especially PLGA and PLA, polycaprolactone (PCL);
the material can undergo slight shrinkage, after sealing, in order to follow the shape of the encased tablets in the best possible manner;
thickness of the material: especially from 30 to 300 μm.

The edges of the sheets are to be sealed together over the entire periphery, and the sealed edges are preferably turned down as close to the form as possible so as not to create a relief which would be detrimental to pleasant ingestion (mouth) or to easy swallowing (stomach, intestine). If necessary, before the edges are turned down, they can be made smaller by cutting.

The method of sealing the edges is adapted to the material of the sheets.

According to a fifth embodiment, which is suitable for tablets, the wrapping is a film coating present over the entire outer surface of the tablets brought together by bonding. The tablets have a planar surface, preferably complementary to that of the associated tablet. If three tablets are combined, the middle tablet has two planar surfaces.

In that case, the pharmaceutical dosage form comprises at least two tablets brought together by a separating film coating or bonding and an encasing film coating, the two film coatings advantageously being continuous material. The two film coatings can be of the same material or of materials that are different but are advantageously compatible in order to ensure continuity of material. Conventional film-coating compositions and methods can be used to produce them.

According to a first form, the tablets are wholly film-coated and the film-coated tablets are bonded to one another by a surface that is sufficient to ensure a unitary configuration until they are dissolved in the body cavity.

According to a second form, the tablets are first bonded together and then the whole is film-coated.

The method preferably comprises the separate film-coating of the two tablets, for example by spraying of a film-forming solution. That solution can comprise a copolymer of polyvinyl alcohol and polyethylene glycol. Advantageously, the wrapping and especially the one obtained from said copolymer is rapidly or instantaneously solubilised in aqueous environment at the time the dosage form is ingested. Preferably, the film-forming solution does not modify the solubility parameters of the original tablets. Each tablet can advantageously be film-coated with the aid of its own coloured solution so that the presence of the different tablets is visible to the naked eye.

Bonding can then be carried out with the aid of a suitable bonding solution. That solution can be of the same nature as the film-forming solution, the dilution or viscosity optionally being different. The solution can be of a different nature and comprise other suitable monomers or polymers. The bonding solution can be an aqueous solution or a solution obtained by using a gas maintained in the supercritical liquid state, such as, for example, carbon dioxide. Bonding itself can be obtained, for example, by simple contact or by fusion by compression, or by another known method.

Bonding can also be effected with the aid of a pulverulent substance placed between the tablets, followed by fusion by compression.

The tablets or coated tablets combined in the same form can have one, a plurality or all of the following features:
if they are tablets and/or coated tablets, they each have a face that is complementary in terms of form with a face of the other, the two faces being intended to be opposite one another in the final pharmaceutical dosage form; those two faces are preferably planar or substantially planar;
the tablets and/or coated tablets each have a planar or substantially planar face and the two faces have the same geometry;
the two complementary faces have a round or ovoid/oval geometry;
the final pharmaceutical dosage form substantially forms a sphere or an elongated sphere (oblong form);
the tablets/coated tablets have substantially identical weights;
the tablets/coated tablets have different weights.

According to a feature of the invention, the geometric form of the medicament is adapted to the needs of the invention. If an existing medicament (reference proprietary medicine) is used, the adaptation is made without changing the qualitative and quantitative composition of the original medicament (“similar galenical form”). Accordingly especially, starting from the active principle and the excipient(s) of the reference proprietary medicine, tablets having the desired form are produced. The two tablets to be combined are produced so that they have the optimum complementary geometric forms described in this specification.

The invention relates also to oral pharmaceutical combinations which are presented in the pharmaceutical dosage form according to the invention and the purpose of which is to propose a therapeutic treatment method permitting the joint administration of medicaments (especially two, three or four medicaments), in which an oral pharmaceutical dosage form according to the invention is administered to a patient who needs it. The various pharmaceutical dosage forms mentioned above can be the subject of that method.

According to a feature of the invention, the doses of the medicaments so combined are coordinated. This means that the dosages of the various combined medicaments correspond to the medical prescription which defines the respective doses of each of the medicaments for optimum therapeutic effectiveness in a single administration.

More particularly, the invention relates to an oral pharmaceutical dosage form containing at least two medicaments, in which form the medicaments on the one hand are brought together in the water-soluble wrapping and on the other hand are separated so that they do not come into contact with one another, at least one of the medicaments being selected from the following therapeutic classes: non-steroidal anti-inflammatory drug (NSAID), proton pump inhibitor (PPI), beta-blocker, statin, conversion enzyme inhibitor (CEO, biguanide, myorelaxant, calcium inhibitor, corticoid, antidepressant, benzodiazepine, non-atropine-like intestinal transit retarder, intestinal antibacterial, and from the following therapeutic molecules: spironolactone, aldactone, propranolol, clarithromycin, amoxycillin, low-dose acetylsalicylic acid (aspirin), carvedilol, potassium, clopidogrel.

The invention relates especially to combinations of two, three or four medicaments.

The medicaments can be chosen to constitute the following preferred combinations:
a non-steroidal anti-inflammatory drug (NSAID) combined with a proton pump inhibitor (PPI);
a beta-blocker combined with a statin;
a conversion enzyme inhibitor (CEI) combined with a statin;
a statin combined with a biguanide;
an NSAID combined with a myorelaxant;
a calcium inhibitor combined with a CEI;
spironolactone combined with propranolol;
a PPI combined with clarithromycin and amoxycillin;
low-dose acetylsalicylic acid (“infant-type” dose, which has an effect of fluidising the blood in adult patients and is commonly used in cardiology to treat problems of hypertension; dose from 75 mg, for example from 75 to 160 mg dose) combined with clopidogrel;
low-dose acetylsalicyclic acid combined with a CEI, a statin and a beta-blocker;
a corticoid (doses varying from 20 to 100 mg especially) combined with potassium;
an antidepressant combined with a benzodiazepine (anxiolytic);
a non-atropine-like intestinal transit retarder combined with an intestinal antibacterial.

The invention relates to an oral pharmaceutical dosage form containing a medicament comprising an NSAID and a medicament comprising a PPI.

The indication is the anti-inflammatory treatment of patients at risk of gastroduodenal lesions.

The invention relates also to the use of a medicament comprising an NSAID and of a medicament comprising a PPI in the preparation of a two-in-one oral pharmaceutical dosage form according to the invention as an anti-inflammatory for patients at risk of gastroduodenal lesions.

As NSAID there may be mentioned especially, without being limited: naproxen, ketoprofen, diclofenac, ibuprofen.

As PPI there may be mentioned especially, without being limited: omeprazole, esomeprazole and lansoprazole.

The invention relates also to an oral pharmaceutical dosage form containing a medicament comprising a beta-blocker and a medicament comprising a statin.

The indication is hypercholesterolaemia in patients with arterial hypertension.

The invention relates also to the use of a medicament comprising a beta-blocker and of a medicament comprising a statin in the preparation of a two-in-one oral pharmaceutical dosage form according to the invention for the treatment of hyperchloesterolaemia in patients with arterial hypertension.

As beta-blocker there may be mentioned especially, without being limited: bisoprolol, atenolol, acebutolol.

As statin there may be mentioned especially, without being limited: pravastatin, simvastatin.

The invention relates also to an oral pharmaceutical dosage form containing a medicament comprising a CEI and a medicament comprising a statin.

The indication is the prevention of cardiovascular disorders in at-risk subjects, especially in arterial hypertension associated with hypercholesterolaemia.

The invention relates also to the use of a medicament comprising a CEI and of a medicament comprising a statin in the preparation of a two-in-one oral pharmaceutical dosage form according to the invention for the prevention of cardiovascular disorders in at-risk subjects, especially in arterial hypertension associated with hypercholesterolaemia.

As statin there may be mentioned especially, without being limited: pravastatin, simvastatin.

As CEI there may be mentioned especially, without being limited: ramipril, captopril, enalapril and fosinopril.

The invention relates also to an oral pharmaceutical dosage form containing a medicament comprising a biguanide and a medicament comprising a statin.

The indication is the prevention of cardiovascular risks in patients with type 2 diabetes.

The invention relates also to the use of a medicament comprising a biguanide and of a medicament comprising a statin in the preparation of a two-in-one oral pharmaceutical dosage form according to the invention for the prevention of cardiovascular risks in patients with type 2 diabetes.

As statin there may be mentioned especially, without being limited: pravastatin, simvastatin.

As biguanide there may be mentioned especially, without being limited: metformin, gliclazide, carbutamide and glibenclamide.

The invention relates also to an oral pharmaceutical dosage form containing a medicament comprising an NSAID and a medicament comprising a myorelaxant.

The indication is the treatment of common acute lumbar pain.

The invention relates also to the use of a medicament comprising an NSAID and of a medicament comprising a myorelaxant in the preparation of a two-in-one or three-in-one oral pharmaceutical dosage form according to the invention for the treatment of common acute lumbar pain.

As myorelaxant there may be mentioned especially, without being limited: thiocolchicoside.

As NSAID there may be mentioned especially, without being limited: naproxen, ketoprofen, diclofenac, ibuprofen.

The invention relates also to an oral pharmaceutical dosage form containing a medicament comprising a calcium inhibitor and a medicament comprising a CEI.

The indication is the treatment of arterial hypertension resistant to monotherapy.

The invention relates also to the use of a medicament comprising a calcium inhibitor and of a medicament comprising a CEI in the preparation of a two-in-one oral pharmaceutical dosage form according to the invention for the treatment of hypertension resistant to monotherapy,

As CEI there may be mentioned especially, without being limited ramipril, captopril, enalapril and fosinopril.

As calcium inhibitor there may be mentioned especially, without being limited nifedipine, felodipine and nicardipine.

The invention relates also to an oral pharmaceutical dosage form containing a medicament comprising spironolactone and a medicament comprising propranolol.

The indication is the treatment of cirrhosis in patients with pulmonary arterial hypertension.

The invention relates also to the use of a medicament comprising spironolactone and of a medicament comprising propranolol in the preparation of a two-in-one oral pharmaceutical dosage form according to the invention for the treatment of cirrhosis in patients with pulmonary arterial hypertension.

The invention relates also to an oral pharmaceutical dosage form containing a medicament comprising a PPI, a medicament comprising clarithromycin and a medicament comprising amoxycillin.

The indication is the eradication treatment of the bacterium Helicobacter pylori in the case of gastroduodenal ulcer disease in adults; proof of lesion and infection is usually provided by endoscopy.

The invention relates also to the use of a medicament comprising a PPI, of a medicament comprising clarithromycin and of a medicament comprising amoxycillin in the preparation of a three-in-one oral pharmaceutical dosage form according to the invention for the eradication of the bacterium Helicobacter pylori in the case of gastroduodenal ulcer disease in adults.

As PPI there may be mentioned especially, without being limited: omeprazole, esomeprazole and lansoprazole.

The invention relates also to an oral pharmaceutical dosage form containing a medicament comprising low-dose acetylsalicylic acid, a medicament comprising a CEI, a medicament comprising a statin and a medicament comprising a beta-blocker.

The indication is post-infarct treatment (with or without cardiac insufficiency).

The invention relates also to the use of a medicament comprising low-dose acetylsalicylic acid, of a medicament comprising a CEI, of a medicament comprising a statin and of a medicament comprising a beta-blocker in the preparation of a four-in-one oral pharmaceutical dosage form according to the invention for post-infarct treatment.

As beta-blocker there may be mentioned especially, without being limited: bisoprolol, atenolol, acebutolol, carvedilol.

As statin there may be mentioned especially, without being limited: pravastatin, simvastatin.

As CEI there may be mentioned especially, without being limited ramipril, captopril, enalapril and fosinopril.

The invention relates also to an oral pharmaceutical dosage form containing a medicament comprising low-dose acetylsalicylic acid and a medicament comprising clopidogrel.

The indication is the prevention of the risk of thromboembolism in hypertensive patients and in patients who have undergone angioplasty.

The invention relates also to the use of a medicament comprising low-dose acetylsalicyclic acid and of a medicament comprising clopidogrel in the preparation of a two-in-one oral pharmaceutical dosage form according to the invention for the prevention of the risk of thromboembolism in hypertensive patients and in patients who have benefited from an angioplasty.

The invention relates also to an oral pharmaceutical dosage form containing a medicament comprising a corticoid and a medicament comprising potassium.

The indication is the prevention of hypokaliaemia in patients who are to be treated by corticotherapy.

The invention relates also to the use of a medicament comprising a corticoid and of a medicament comprising potassium in the preparation of a two-in-one oral pharmaceutical dosage form according to the invention for the prevention of hypokaliaemia in patients who are to be treated by corticotherapy.

The corticoid can be used in doses varying from 20 to 100 mg.

As corticoid there may be mentioned especially, without being limited: prednisolone, prednisone.

Potassium is generally in the form of a potassium salt. As potassium salt there may be mentioned especially potassium chloride.

The invention relates also to an oral pharmaceutical dosage form containing a medicament comprising an antidepressant and a medicament comprising a benzodiazepine.

The indication is the treatment of depression.

The invention relates also to the use of a medicament comprising an antidepressant and of a medicament comprising a benzodiazepine in the preparation of a three-in-one oral pharmaceutical dosage form according to the invention for the treatment of depression.

The antidepressant can be especially a tricyclic antidepressant, for example clomipramine, or a selective serotonin reuptake inhibitor, for example fluoxetine or venlafaxine.

The benzodiazepine (anxiolytic) can be, without being limited bromazepan, alprazolam.

The invention relates also to an oral pharmaceutical dosage form containing a medicament comprising a non-atropine-like intestinal transit retarder and a medicament comprising an intestinal antibacterial.

The indication is the reestablishment of intestinal transit and treatment of bacterial diarrhoea.

The invention relates also to the use of a medicament comprising a non-atropine-like intestinal transit retarder and of a medicament comprising an intestinal antibacterial in the preparation of a two-in-one oral pharmaceutical dosage form according to the invention for reestablishing intestinal transit and the treatment of bacterial diarrhoea.

As non-atropine-like intestinal transit retarder there may be mentioned fort example loperamide.

As intestinal antibacterial there may be mentioned, for example, nifuroxazide.

For each of the precise combinations which have just been described, the medicaments are preferably brought together in a water-soluble wrapping and are separated so that their active principles cannot come into contact with one another.

The medicaments mentioned within the context of those various combinations can be in the various galenical forms mentioned above.

The medicaments according to the invention are or can be the subject of commercial forms which can be used as such for implementing the invention. Among the commercial forms or reference specialities which can be used there may be mentioned, for example:
Elisor® (pravastatin), Zocor® (simvastatin) for the statins (class C10A1)
Mopral® (omeprazole) for the proton pump inhibitor (class A2B2);
Naprosyne® (naproxen), Nurofen® (ibuprofen), Ketum® (ketoprofen) and Voltaren® (diclofenac) for the non-steroidal antiinflammatory drugs (M1A1);
Detensiel® or Soprol® (bisoprolol), Tenormine® (atnolol), Sectral® (acebutolol) for the beta-blockers (class C7A); Kredex® (carvedilol);
Triatec® (ramipril), Lopril® (captopril), Renitec® (enalapril), Fositec® (fosinopril) for the conversion enzyme inhibitors (CEI, class C9A);
Glucophage® (metformin) and Diamicron® (gliclazide) for the biguanides (class A10B2);
Solupred®, Cortancyl®, Medrol® (corticoids);
Kaleorid®, Difuca® (potassium salt);
Zeclar® or Naxy® (clarithromycin), Clamoxyl or Agram (amoxycillin);
Aldactone® 50 (spironolactone), Avlocardyl® 40 (propranolol);
Adalat® LD (nifedipine), Flodil® (felodipine), Loxen® (nicardipine);
Imossel® and Imodium® (loperamide), Ercefuryl®(nifuroxozide);
Plavix® (clopidogrel);
Anafranil (clomipramine), Prozac® (fluoxetine), Effexor® (venlafaxine);
Lexomil® (bromazepan), Xanax® (alprazolam)®;
or their generic proprietary medicine

However, according to one feature of the invention, the geometric form of the medicaments can be adapted to the needs of the invention. If an existing medicament (reference generic proprietary medicine) is used, the adaptation is made without changing the qualitative and quantitative composition of the original medicament (“similar galenical form”). Accordingly especially, starting from the active principle and the excipient(s) of the reference proprietary medicine, tablets having the desired form are produced. The two tablets to be combined are produced so that they have the optimum complementary geometric forms described in this specification.

The invention relates also to a therapeutic treatment method which permits the joint administration of medicaments (especially two, three or four medicaments), in which an oral pharmaceutical dosage form according to the invention is administered to a patient who needs it, for the indicated therapeutic indication. The various pharmaceutical dosage forms mentioned above and their therapeutic indications as presented in this specification can be the subject of this method.

According to one feature of the invention, the medicaments have coordinated therapeutic dosage. This means that the dosages of the associated medicaments are optimised according to an optimum therapeutic effectiveness in a single administration.

A first embodiment accordingly consists in administering an oral pharmaceutical dosage form containing a medicament comprising an NSAID and a medicament comprising a PPI. The indication is anti-inflammatory treatment in patients at risk of gastroduodenal lesions. Medicaments and combinations which can be used have been described supra.

A second embodiment consists in administering an oral pharmaceutical dosage form containing a medicament comprising a beta-blocker and a medicament comprising a statin. The indication is hypercholesterolaemia in patients with arterial hypertension. Medicaments and combinations which can be used have been described supra.

A third embodiment consists in administering an oral pharmaceutical dosage form containing a medicament comprising a CEI and a medicament comprising a statin. The indication is the prevention of cardiovascular disorders in at-risk subjects, especially in the case of arterial hypertension associated with hypercholesterolaemia. Medicaments and combinations which can be used have been described supra.

A fourth embodiment consists in administering an oral pharmaceutical dosage form containing a medicament comprising a biguanide and a medicament comprising a statin. The indication is the prevention of cardiovascular risks in patients with type 2 diabetes. Medicaments and combinations which can be used have been described supra.

A fifth embodiment consists in administering an oral pharmaceutical dosage form containing a medicament comprising an NSAID and a medicament comprising a myorelaxant. The indication is the treatment of common acute lumbar pain. Medicaments and combinations which can be used have been described supra.

A sixth embodiment consists in administering an oral pharmaceutical dosage form containing a medicament comprising a calcium inhibitor and a medicament comprising a CEI. The indication is the treatment of arterial hypertension resistant to monotherapy. Medicaments and combinations which can be used have been described supra.

A seventh embodiment consists in administering an oral pharmaceutical dosage form containing a medicament comprising spironolactone and a medicament comprising propranolol. The indication is the treatment of cirrhosis in patients with pulmonary arterial hypertension. Medicaments and combinations which can be used have been described supra.

An eighth embodiment consists in administering an oral pharmaceutical dosage form containing a medicament comprising a PPI, a medicament comprising clarithromycin and a medicament comprising amoxycillin. The indication is the eradication treatment of the bacterium Helicobacter pylori in the case of gastroduodenal ulcer disease in adults; proof of lesion and infection is usually provided by endoscopy. Medicaments and combinations which can be used have been described supra.

A ninth embodiment consists in administering an oral pharmaceutical dosage form containing a medicament comprising low-dose acetylsalicylic acid, a medicament comprising a CEI, a medicament comprising a statin and a medicament comprising a beta-blocker. The indication is post-infarct treatment (with or without cardiac insufficiency). Medicaments and combinations which can be used have been described supra.

A tenth embodiment consists in administering an oral pharmaceutical dosage form containing a medicament comprising low-dose acetylsalicylic acid and a medicament comprising clopidogrel. The indication is the prevention of the risk of thromboembolism in hypertensive patients and in patients who have undergone angioplasty. Medicaments and combinations which can be used have been described supra.

An eleventh embodiment consists in administering an oral pharmaceutical dosage form containing a medicament comprising a corticoid and a medicament comprising potassium. The indication is the prevention of hypokaliaemia in patients requiring treatment by corticotherapy. Medicaments and combinations which can be used have been described supra.

A twelfth embodiment consists in administering an oral pharmaceutical dosage form containing a medicament comprising an antidepressant and a medicament comprising a benzodiazepine. The indication is the treatment of depression. Medicaments and combinations which can be used have been described supra.

A thirteenth embodiment consists in administering an oral pharmaceutical dosage form containing a medicament comprising a non-atropine-like intestinal transit retarder and a medicament comprising an intestinal antibacterial. The indication is the reestablishment of intestinal transit and the treatment of bacterial diarrhoea. Medicaments and combinations which can be used have been described supra.

The present invention relates also to a process for producing oral pharmaceutical dosage forms according to the invention. The process comprises a step of bringing the two or more medicaments together in the leakproof water-soluble wrapping and optionally a step of separating the medicaments by interposing a sheet or wall; that step preceding or being simultaneous with the step of bringing together.

Details relating to the production of each of the embodiments of the pharmaceutical dosage forms according to the invention are given in the remainder of the description.

A feature of the process is the bringing together of two or more preconstituted, especially existing or commercial, medicaments or galenical forms.

According to another feature, the format of at least one of the medicaments is modified in order to adapt it to the format of the other in order to produce a pharmaceutical dosage form that is as compact as possible and easy to ingest. Especially, the formats of the two or more medicaments are adapted.

For example, the tablets are chosen or formatted to have planar surfaces opposite one another. They are also chosen or formatted to have planar surfaces whose sizes and forms are similar, or even better, identical.

The invention will now be described with the aid of embodiments given by way of non-limiting example and with reference to the accompanying drawings.

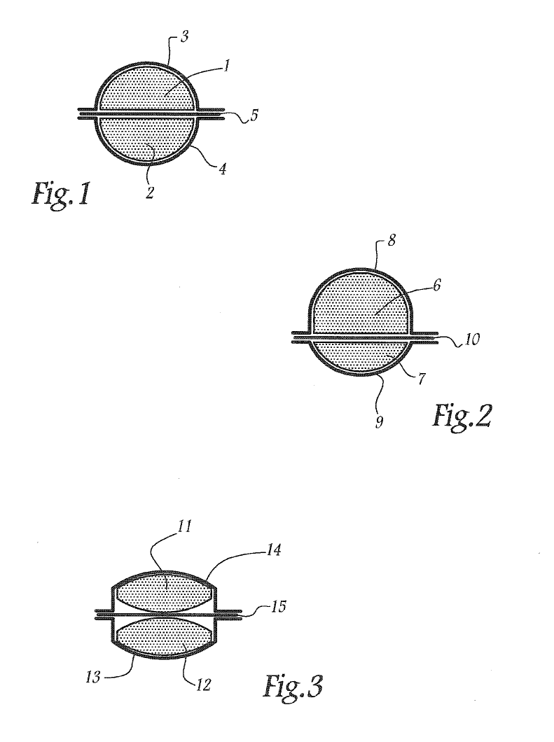

FIGS. 1, 2 and 3 show three cross-sectional representations, in diagrammatic form, of pharmaceutical dosage forms according to the invention, using sheets of pharmaceutical material and prior to sealing of the edges.

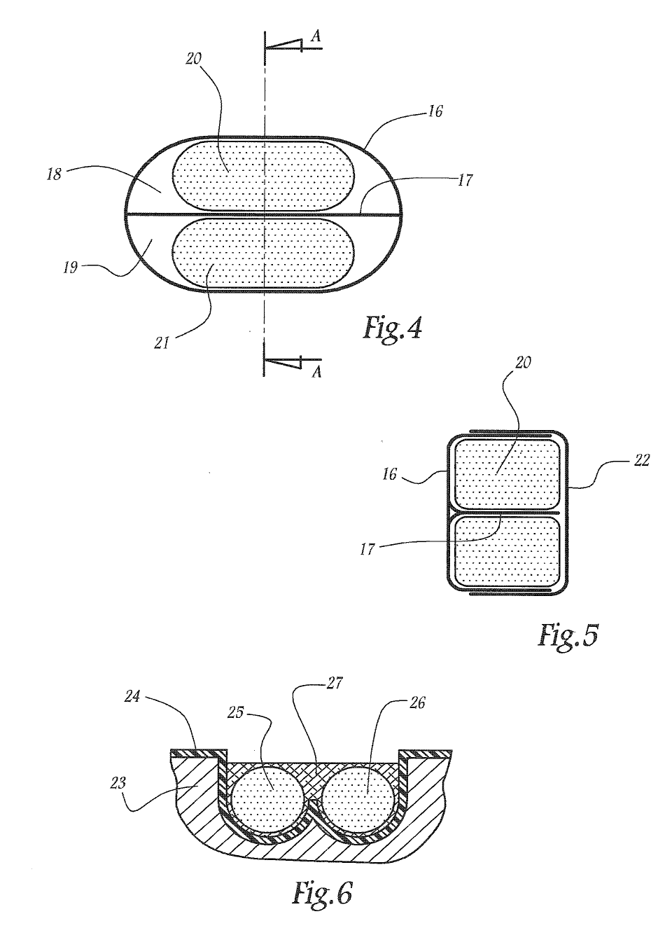

FIGS. 4 and 5 are cross-sectional representations, in diagrammatic form, of an embodiment using a matrix of rice paper.

FIG. 6 shows, in diagrammatic form, an embodiment by inclusion in a water-soluble matrix.

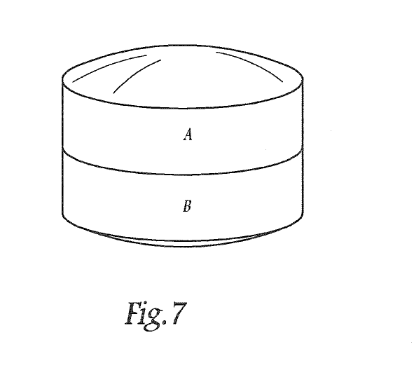

FIG. 7 shows, in diagrammatic form, an embodiment of two tablets associated using a coating.

FIG. 1 shows a pharmaceutical dosage form comprising two tablets 1 and 2 having the same hemispherical form and having the same volume, for example 200 mg each. Combination to give a substantially spherical single pharmaceutical dosage form is obtained by using 3 sheets of pharmaceutical film of cellulose copolymer, denoted by the reference numerals 3, 4 and 5, reference numeral 5 denoting the separating sheet between the planar faces of the tablets 1 and 2. For the three embodiments shown, the edges of the three sheets are to be sealed together over the entire periphery, and the sealed edges are turned down as close to the form as possible so as not to create a relief which is detrimental to easy swallowing. If necessary, the edges can be made smaller by cutting before they are turned down.

By way of variation, two commercially available tablets are used without modifying their form.

FIG. 2 shows a pharmaceutical dosage form comprising two tablets 6 and 7 having different volumes, for example 400 mg and 200 mg, respectively. Combination to give a substantially spherical single pharmaceutical dosage form is obtained using 3 polymer sheets, for example of polyvinyl alcohol, denoted by the reference numerals 8, 9 and 10, reference numeral 10 denoting the separating sheet between the planar faces of the tablets 6 and 7.

By way of variation, two commercially available tablets are used without modifying their form.

FIG. 3 shows a pharmaceutical dosage form comprising two tablets 11 and 12 having the same form and having the same volume, for example 300 mg each. Combination to give a single pharmaceutical dosage form is obtained using 3 polycaprolactone sheets denoted by the reference numerals 13, 14 and 15, reference numeral 15 denoting the separating sheet between the planar faces of the tablets 11 and 12.

FIG. 4 shows the body of a matrix of rice paper comprising an outer wrapping 16 and a separating wall 17, the whole delimiting two compartments 18 and 19. Each of the compartments of the body is shown containing a galenical form 20, 21. The matrix comprises a flat base (not shown) and a cover (not shown). FIG. 5 shows a cross-sectional view according to AA of FIG. 4, in which there is this time shown not only the body of the matrix but also its cover 22. The cover 22 is fitted onto the body of the matrix. It will be noted that the matrix body, as well as the cover, can be made in the conventional manner, by moulding a rice paper solution and curing.

FIG. 6 shows a steel mould 23, a blister cell 24 of PVC or PVC-PVDC or of any other alimentary plastics material suitable for forming a blister cell. This cell may or may not be coated with one or more protective films. It can simply be thermoformed in the mould 23. The two medicaments 25 and 26 are placed in the cell, and then the binder, which will form the inclusion matrix 27, is poured in. The matrix is then cured by cooling. If the matrix is made of gelatin, drying is also provided. If the matrix is made of PEG, for example PEG 6000, curing is simply carried out by cooling. The cell is then closed by a sealing sheet, conventionally by thermosealing of an aluminium complex (not shown).

FIG. 7 shows a pharmaceutical dosage form obtained by a process of film-coating and bonding. Each of the tablets A and B is film-coated individually in a revolving drum by spraying of a film-forming solution obtained, for example, from a copolymer of polyvinyl alcohol and polyethylene glycol. The spraying solutions have a different colour for each of the tablets.

Bonding is then carried out by deposition of a solution obtained with the film-forming substance. The concentration of the bonding solution is adjusted, if necessary, in order to have a suitable viscosity. Bonding is carried out by simple contact or by compression fusion.

The present specification focuses substantially on the combination of two medicaments. The person skilled in the art will easily be able to adapt the pharmaceutical dosage form to the presence of more than two medicaments, especially three or four medicaments, by incorporating as many separating sheets or walls as are necessary. It will be possible for the person skilled in the art to envisage pharmaceutical dosage forms other than those which have just been described. For example, he will be able to employ gelatin capsules, and in particular multi-compartment gelatin capsules, without the disadvantages associated with the difficulties of filling and handling that are encountered in the prior art, because a medicament within the scope of the invention, that is to say a solid oral galenical form, is placed in each compartment. It will then be possible, for example, to assemble the parts of gelatin capsules on a horizontal plane and under industrially acceptable conditions. Such a gelatin capsule therefore comprises at least two compartments each containing a medicament according to the invention, which are separated from one another by a separating wall.

What is claimed is:

1. An oral pharmaceutical dosage form comprising at least two medicaments, wherein the medicaments are in the form of separately preconstituted tablets, wherein the preconstituted tablets are film coated and the film coating is present over the entire outer surface of the preconstituted tablets and wherein the preconstituted tablets are bonded together.

2. The pharmaceutical dosage form according to claim 1, wherein the medicaments comprise commercial galenical forms and/or galenical forms having the same qualitative and quantitative composition as commercial galenical forms but a different geometric form.

3. The pharmaceutical dosage form according to claim 1, wherein the film coating comprises a material that is water-soluble at the pH of the mouth, at the pH of the stomach or at the pH of the intestine.

4. The pharmaceutical dosage form according to claim 1, comprising two, three or four medicaments.

5. The pharmaceutical dosage form according to claim 1, wherein the total weight of medicament is less than or equal to 1500 mg.

6. The pharmaceutical dosage form according to claim 1, wherein the film coatings are applied using a film coating solution, wherein the film coatings of the different tablets are identical or different, and wherein either the tablets are first film-coated and than bonded or are first bonded and then film-coated.

7. The pharmaceutical dosage form according to claim 1 wherein the film coating is formed of or comprises polyvinyl alcohol.

8. The pharmaceutical dosage form according to claim 1, wherein the tablets each have a planar surface and the bonding is performed between the planar surfaces.

9. The pharmaceutical dosage form according to claim 8, wherein the planar surfaces of the tablets are complementary.

10. The pharmaceutical dosage form according to claim 1, wherein the film coating comprises a copolymer of polyvinyl alcohol and polyethylene glycol.

11. The pharmaceutical dosage form according to claim 1, wherein the film coating is applied using a film coating solution and wherein the tablets are bonded using a bonding solution.

12. The pharmaceutical dosage form according to claim 11, wherein the film coating solution and the bonding solution are the same.

13. The pharmaceutical dosage form according to claim 11, wherein the film coating solution and the bonding solution comprise a copolymer of polyvinyl alcohol and polyethylene glycol.

14. The pharmaceutical dosage form according to claim 1, wherein the combined medicaments are to be absorbed in the same or a different cavity of the digestive system.

15. The pharmaceutical dosage form according to claim 1 wherein at least one of the medicaments is selected from the group consisting of a non-steroidal anti-inflammatory drug (NSAID), a proton pump inhibitor (PPI), a beta-blocker, statin, a conversion enzyme inhibitor (CEI), biguanide, a myorelaxant, a calcium inhibitor, corticoid, an antidepressant, benzodiazepine, a non-atropine-like intestinal transit retarder, an intestinal antibacterial, and therapeutic molecules selected from the group consisting of spironolactone, propranolol, clarithromycin, amoxycillin, low-dose acetylsalicylic acid, potassium, and clopidogrel.

16. The pharmaceutical dosage form according to claim 15 wherein the medicaments are selected from the group consisting of the following combinations:
a non-steroidal anti-inflammatory drug (NSAID) combined with a proton pump inhibitor (PPI);
a beta-blocker combined with a statin;
a conversion enzyme inhibitor (CEI) combined with a statin;
a statin combined with a biguanide;
an NSAID combined with an analgesic and/or a myorelaxant;
a calcium inhibitor combined with a CEI;
spironolactone combined with propranolol;
a PPI combined with clarithromycin and amoxycillin;
low-dose acetylsalicyclic acid combined with a CEI, a statin and a beta-blocker;
low-dose acetylsalicylic acid combined with clopidogrel;
a corticoid combined with potassium;
an antidepressant combined with a benzodiazepine; and
a non-atropine-like intestinal transit retarder combined with an intestinal antibacterial.

17. The pharmaceutical dosage form according to claim 16 comprising
a medicament comprising an NSAID and a medicament comprising a PPI, for the anti-inflammatory treatment of patients at risk of gastroduodenal lesions; or
a medicament comprising a beta-blocker and a medicament comprising a statin, for the treatment of hypercholesterolaemia in patients with arterial hypertension; or
a medicament comprising a CEI and a medicament comprising a statin, for the prevention of cardiovascular disorders in at-risk subjects, especially in the case of arterial hypertension associated with hypercholesterolaemia; or
a medicament comprising a biguanide and a medicament comprising a statin, for the prevention of cardiovascular risks in patients with type 2 diabetes; or
a medicament comprising an NSAID and a medicament comprising a myorelaxant, for the treatment of common acute lumbar pain; or
a medicament comprising a calcium inhibitor and a medicament comprising a CEI, for the treatment of arterial hypertension resistant to monotherapy; or
a medicament comprising spironolactone and a medicament comprising propranolol, for the treatment of cirrhosis in patients with pulmonary arterial hypertension; or
a medicament comprising a PPI, a medicament comprising clarithromycin and a medicament comprising amoxycillin, for the eradication treatment of the bacterium Helicobacter pylori in the case of gastroduodenal ulcer disease in adults, especially following endoscopic proof of lesion and infection; or
a medicament comprising low-dose acetylsalicylic acid, a medicament comprising a CEI, a medicament comprising a statin and a medicament comprising a beta-blocker, for post-infarct treatment; or
a medicament comprising low-dose acetylsalicylic acid and a medicament comprising clopidogrel, for the prevention of the risk of thromboembolism in hypertensive patients and in patients who have undergone angioplasty; or
a medicament comprising a corticoid and a medicament comprising potassium, for the prevention of hypokaliaemia in patients requiring treatment by corticotherapy; or
a medicament comprising an antidepressant, a medicament comprising a benzodiazepine, for the treatment of depression; or
a medicament comprising a non-atropine-like intestinal transit retarder and a medicament comprising an intestinal antibacterial, for the reestablishment of intestinal transit and the treatment of bacterial diarrhoea.

18. The pharmaceutical dosage form according to claim 16, wherein
the biguanide is selected from the group consisting of metformin, gliclazide, carbutamide and glibenclamide; or
the myorelaxant is thiocolchicoside; or
the calcium inhibitor is selected from the group consisting of nifedipine, felodipine and nicardipine; or
potassium is a potassium salt; or
the corticoid is selected from the group consisting of prednisolone and prednisone; or
the non-atropine-like intestinal transit retarder is loperamide; or
the antibacterial is nifuroxazide; or
the NSAID is selected from the group consisting of naproxen, ketoprofen, diclofenac and ibuprofen; or
the CEI is selected from the group consisting of ramipril, captopril, enalapril and fosinopril; or
the PPI is selected from the group consisting of omeprazole, esomeprazole and lansoprazole; or
the statin is selected from the group consisting of pravastatin and simvastatin; or
the beta-blocker is selected from the group consisting of bisoprolol, atenolol, acebutolol, and carvedilol; or
the antidepressant is a tricyclic antidepressant or a selective serotonin reuptake inhibitor; or
the benzodiazepine is selected from the group consisting of bromazepan and alprazolam.

19. The pharmaceutical dosage form according to claim 18, wherein the antidepressant is clomipramine.

20. The pharmaceutical dosage form according to claim 18, wherein the selective serotonin reuptake inhibitor is fluoxetine or venlafaxine.
